(19) United States
(12) Patent Application Publication
(10) Pub. No.: US 2006/0057077 A1
(43) Pub. Date: Mar. 16, 2006

(54) Dental care product

(75) Inventor: Maurice John Adward White, Wantirna (AU)

Correspondence Address: CONNOLLY BOVE LODGE & HUTZ LLP, SUITE 800, 1990 M STREET NW, WASHINGTON, DC, 20036-3425, US

(73) Assignee: YNDK Pty Ltd, Wantirna South (AU)

(21) Appl. No.: 10/531,306

(22) PCT Filed: Oct. 28, 2003
(86) PCT No.: PCT/AU03/01411

§ 371 Date: Apr. 13, 2004

(30) Foreign Application Priority Data
Oct. 29, 2002 (AU) 2002952305

(51) Int. Cl. A61K 8/00; A61C 5/00

(52) U.S. Cl. 424/49; 433/217.1

(57) ABSTRACT

A masticating strip useful in dental hygiene consisting of a cellular foam in which the surface cells incorporate longitudinal slits. The strips are sized to give a chewing volume of 1 to 4 cm³ preferably about 3 cm³. The strips are made from a foam of a thermoplastic material selected from one or more of polyethylene (PE), polyvinyl acetate (PVA) or polyisobutylene. The strips may incorporate a dental adjuvant or a barrier material.

### BACKGROUND TO THE INVENTION

It is known that chewing produces saliva which is a natural means of removing caries causing products from the teeth.
U.S. Pat. Nos. 5,114,704 and 6,178,922 disclose chewable products for dog dental care.
U.S. Pat. No. 5,133,971 proposes using a chewable membrane of reconstituted cellulose.
U.S. Pat. No. 4,554,154 discloses the use of a chewable tape carrying an adhesive.
U.S. Pat. No. 4,891,209 discloses a latex rubber masticating block [chewing gum size] which is intended to inhibit caries.
U.S. Pat. No. 5,939,049 discloses a chewing stick of jute or other natural fibres. It is claimed to be an improvement in the natural chewing sticks used by people in many developing countries.
U.S. Pat. No. 6,123,982 discloses an expandable dental floss and U.S. Pat. No. 6,039,054 to a dental floss having a foamed layer around the core.
Patent specification WO 00/32135 discloses a chewable foam strip of PE or EVA.

The foam cells are closed and smaller than 1 mm in diameter.

A large proportion of caries infections occur in deep cracks and fissures which naturally occur in healthy teeth. There are three areas that are subject to tooth decay, gum margins and between teeth as well as inside pits and fissures. However brushing and flossing, mouth wash, toothpaste and chewing gum can only access and benefit the first 2 areas. They cannot access inside pits and fissures where 80% of cavities occur.

The prior attempts as disclosed in the patents mentioned above do not effectively remove caries forming products from these deep cracks and fissures nor do they prevent those materials from entering the cracks and fissures. The only effective treatment of deep cracks and fissures is to fill or seal them with a permanent barrier material which operation is performed by a dentist at a dental surgery. It is an object of this invention to provide a product which prevents cariogenic food products breaking down into caries producing acid inside these cracks and fissures as well as between teeth and at gum margins by removing them.

### BRIEF DESCRIPTION OF THE INVENTION

To this end the present invention provides a dental care strip of a cellular foam in which the surface cells are open cup shaped cells. The dental care strip can be used as dental floss or as a masticating strip to aid in cleaning teeth or delivering barrier materials to the cracks and fissures where caries most commonly commences.

This invention is predicated on the discovery that the deep cracks and fissures of teeth are not easily penetrated by saliva which is being worked around by chewing gum or closed foam cells. The velocity of the saliva is higher when the foam cells are broken but it takes some time chewing before a sufficient number of cells are broken. This process is expedited by slitting the foam to provide outer cup shaped cells on each foam strip. Half open cells are not as effective as cup shaped cells in propelling saliva with velocity into pits and fissures in the teeth. Preferably the foam cells are large and at least 1 mm in size. Preferably the foam sheet is slit diagonally across the thickness because this not only provides the best shaped open cell to remove plaque and deliver saliva, but also provides a tapered edge that passes between the teeth more effectively. With this invention even 5 mm thick foam strips can be compressed to pass between the teeth.

In assessing the efficacy of various materials to provide sufficient force to the saliva to force it quickly into the deep cracks and fissures a glass model of a fissured tooth has been studied using food dye to indicate penetration. This methodology has reduced the time needed to ascertaining if a procedure will benefit teeth from years (based on clinical trials) to a matter of hours.

Because the cells are cup shaped after being slit they quickly fill with a saliva mix which is forced out as the cup shaped cell is squeezed while chewing. This has been proved by the glass model of a fissure. Foam masticating strips as disclosed in specification WO 00/32135 do not allow sufficient penetration of the deep cracks and fissures within two or three minutes of chewing. Even pin hole fracturing of the cells does not produce sufficient pumping action.

The masticating strips of this invention are sized to give a chewing volume of 1 to 4 cm³ preferably about 2 cm³. The strips may be 50×20×2 mm in dimension with 1 mm foam cells but preferably are 15×2×100 mm.

Any food grade plastic foam may be used including thermoplastics such as polyethylene (PE), polyvinyl acetate (PVA) or polyisobutylene (PIB) to improve cell size and provide thinner cell walls and provide a chewy feel.

The foam strips of this invention may also be combined with barrier materials that are able to penetrate the deep cracks and fissures with the aid of the masticating strips of this invention. These barrier materials are inert and prevent carbohydrate foods from entering the cracks and fissures and prevent the formation of the acids which cause caries. Suitable barrier materials include cocoa, cheese and fine calcium salt particles such as calcium carbonate combined with a carrier like gelatin. It is preferred to use a foam strip that provides a short acting bolus to deliver the barrier materials but not remove them.

To clean the teeth or to remove barrier materials the foam strips of this invention are formed into a long acting bolus.

### DETAILED DESCRIPTION OF THE INVENTION

To assist with understanding this invention, reference will now be made to the drawings in which:

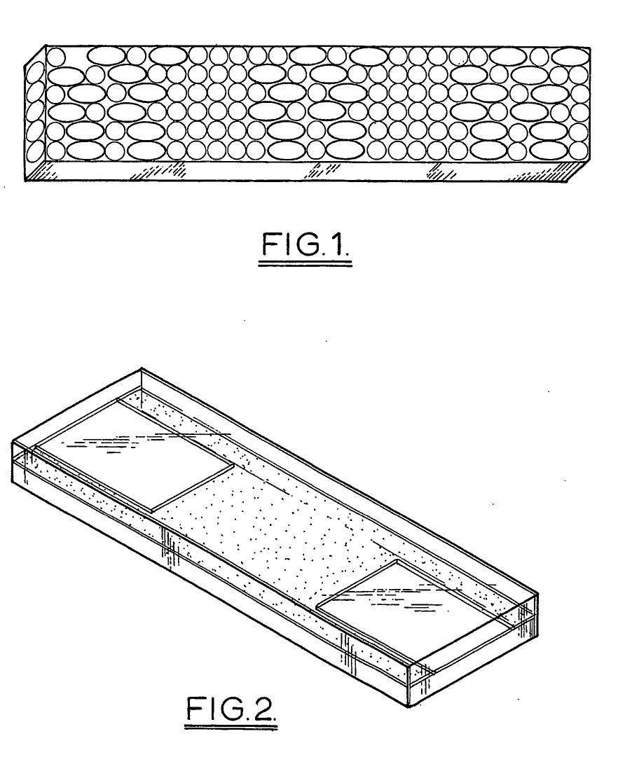

FIG. 1. illustrates a strip of foam of this invention;

FIG. 2. illustrate a glass model which simulates a fissure in a tooth;

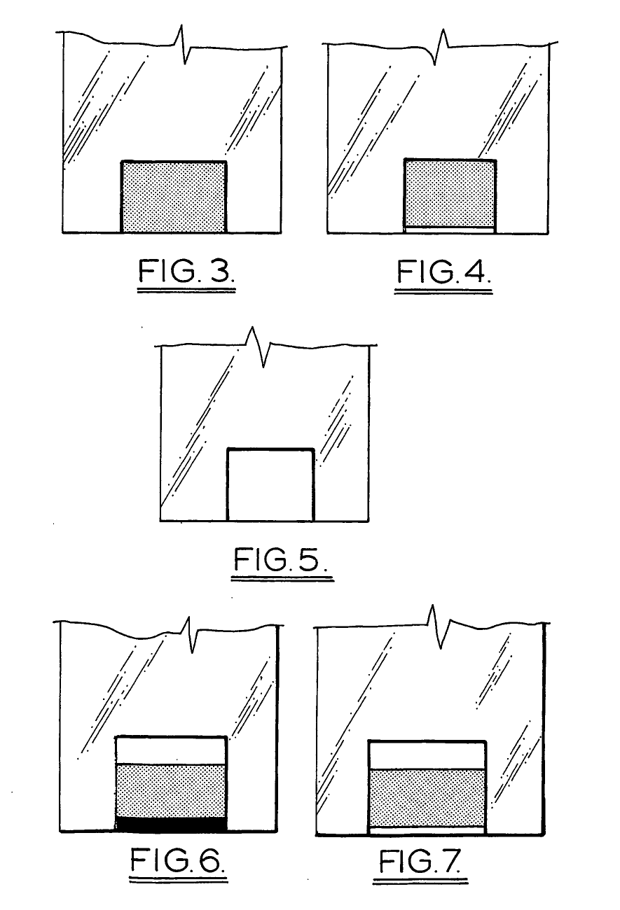

FIGS. 3, 4, 5, 6 and 7. illustrate a fissure at one end of the glass model in FIG. 2. subjected to various foodstuffs and treatments;

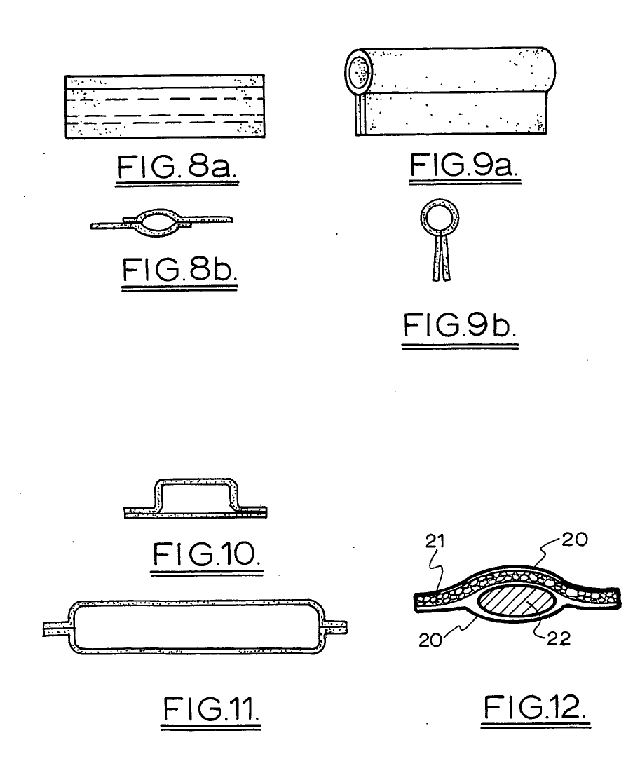

FIGS. 8a and 8b. illustrate a food product which incorporates a dental strip of this invention;

FIGS. 9aand 9b. illustrate another product configuration;

FIG. 10 illustrates another package utilizing the foam of this invention;

FIG. 11 illustrates another package utilizing the foam of this invention.

FIG. 12 illustrates a conventional wrapped confection incorporating the foam of this invention.

A strip of foam of this invention as shown in FIG. 1 is 2 mm×15 mm×100 mm which creates a long acting chewy bolus volume of about 3 cm³

The glass model illustrated in FIG. 2 consists of two strips of glass about 5 mm thick and 20 mm wide and about 60 mm long, stuck together with white silicone in an H fashion or clipped together with a thin Teflon gasket. This leaves a very thin pocket or envelope open at each end as in FIG. 2, replicating the actions observed inside pits and fissures. One end is forced into food or other material and saliva or water usually with a dye to clearly show if the food or other material saliva or water mix is forced inside the fissure by the mix texture or consistency. Suitable fibre added to the mix greatly improves penetration inside pits and fissures and identifies if a stable short or long acting bolus is suitable to fill the pocket as illustrated in FIG. 3. Not every fibre or long acting bolus will force saliva etc inside the fissure. Chewing gum is a long acting bolus, which does not absorb and expel the fluids or mixtures and cannot remove material or dye from the model. FIG. 4 illustrates the results of brushing or chewing gum in penetrating and removing food particles in cracks and fissures and it can be seen that these have little effect. In contrast the foams of this invention provide complete penetration in a short time as illustrated in FIG. 5. when cheeses is applied to the glass model as illustrated in FIG. 6 cheese penetrates and seals the fissure. When normal food is applied to the glass model the results as illustrated in FIG. 7 demonstrate that it is difficult to remove.

The masticating dental foam of this invention may be made as follows:

Polyethylene pellets are melted in an extruder with a fine nucleating powder such as flower of fluoride. 2% polyisobutylene and a foaming agent such as butane propane, natural gas or carbon dioxide are pumped into the melt under controlled pressure to foam the mass as it exits the extruder die. This forms large numbers of interlinked fine wall cells. The pressure should be close to the cell bursting pressure to facilitate creating a soft chewy foam. The foamed sheet may be surface treated with a wire roller or slitting blades to open the foam cells with slits. Preferably the foam exits the die as a tubular sheet about 10 or 20 mm thick most preferably 15 mm thick and is then slit vertically into strips 2 or 3 mm thick. Slitting on an angle through the thickness is a possible alternative as it not only provides cup cells on the cut surface but improves ease of passing between teeth. Preferably the strips are rectangular 15 mm by 50 or 100 mm and 2 mm thick.

Another variation provided by this invention is V shaped strip in which the foam is not fully slit so that two strips are joined at one edge and the V shaped strip may be used to envelop the teeth.

A short acting bolus can be created with 5 mm cube foam blocks formed into jubes with a suitable barrier material. The addition of small amounts of polyisobutylene to the foam improves the foam by forming bigger cells and the foam is more chewy and is slightly tacky. Gelatin and PIB are used to prevent dehydration of the barrier material on the foam strips. The PE foam strips of this invention act as a bulking carrier that can run through a gelatin dip at much higher speeds than as individual units and the coated strands pass up and down a drying, cooling setting tower before being cut and packed.

When slit, the cells provide a cup like surface that can hold saliva or an additive that is easy to force into pits and fissures immediately which is not possible with the smooth surface of a closed cell foam or even with punctured cells which close over under pressure. The exposed slit cell edges of this invention are also more efficient at plaque and food removal, cutting into and holding the plaque or food rather than sliding over it. Even square 5 mm thick foam can be compressed so as to pass between the teeth while applying a slight orthodontic pressure that as well as clean teeth could aid jaw growth and help prevent crowded teeth.

Suitable dental agents such as one mg of fluoride may be incorporated inside the cells of each unit of dental foam to give 5 ppm Fluoride ion in the saliva for about 10 minutes after eating and perhaps before bed. It is desirable not to eat anything for about two hours after chewing the long acting fibre bolus to ensure complete remineralisation so the ideal time for mineralisation with fluoride is before going to bed. This dosage is about 5 kilos of powdered NaF per ton of PE added at the hopper of the extruder as a nucleating agent giving about 30 billion cells in 15,000,000 units of dental foam. It is unlikely that the recommended dose of 1 mg of fluoride per day would be exceeded.

Additives may be applied to the foam by dipping or spraying before the foam is cut into strips as long as the open cells hold the additive.

The foam strip of this invention can be presented for use in a variety of ways. FIGS. 8A and B show two foam strips sealing a barrier food with two side tags. The side tags which are dental strips of this invention may be torn off and used after eating to remove food and neutralise acid. The dental strips of this invention which enclose the barrier food is chewed prior to the meals to seal the cracks and fissures with a barrier material such as cheese. A similar product is illustrated in FIGS. 9A and 9B in which the a barrier food is enclosed within a foam tube according to this invention with two dependent tags that can be torn off and used after eating to remove food.

The foam material of this invention can itself be used as a packaging material for foods or confections that can be used for cleaning teeth after the package is opened. FIG. 10 depicts two strips of foam used to pack and seal a confection or snack.

FIG. 11. Shows two strips of foam used to pack and seal a large confection or multiple units of the chewy device. As many as 50 units can be packed in this way to fit an average business envelope and day packs can be cut from this small bulk pack to serve as both barrier snacks before eating and cleaning device or gum after eating. The end sections of the pack can be used as reusable envelopes to carry a day supply of barrier snacks and cleaning devices even with a preferred toothpaste added.

Alternatively the dental strip of this invention can be combined with the wrapper of a confection as shown in FIG. 12 where a sweet 22 is enclosed in a conventional wrapping 20 and the foam 21 of this invention is also included. After eating the sweet the foam can be chewed so that the saliva generated by chewing the foam flushes the cracks and fissures of the teeth.

From the above it can be seen that this invention provides an effective dental hygiene material that is convenient to use at any time particularly prior to and after eating. Those skilled in the art will realize that the invention can be applied to a variety of applications and the size and volume of the masticating strips can vbe varied as desired. The dental adjuvants that are incorporated into the cells or applied to the surface of the strips may be chosen from among the wide range of materials currently available. Similarly other methods of manufacturing the slit cell foam strips may be employed.

1. A dental care masticating strip comprising a thermoplastic polymeric cellular foam having a total volume of 1 to 4 cubic centimeters the strip having on its surface open cup shaped cells of about 1 mm in diameter.

2. A dental care strip as claimed in claim 1 in which thermoplastic cellular foam material is selected from one or more of polyethylene (PE), polyvinyl acetate (PVA) or polyisobutylene.

3. A dental care strip of a cellular foam in which the surface cells are open cup shaped cells.

4. A dental care strip as claimed in claim 3 in which the foam cells are at least 1 mm in diameter.

5. A dental care strip as claimed in claim 3 sized to give a chewing volume of 1 to 4 cm³ preferably about 3 cm³.

6. A dental care strip as claimed in claim 3 in which the strip is formed form a foam sheet by slitting diagonally across the thickness of the foam sheet.

7. A dental care strip as claimed in claim 3 which is made from a foam of a thermoplastic material selected from one or more of polyethylene (PE), polyvinyl acetate (PVA) or polyisobutylene.

8. A dental care strip according claim 1 which also incorporates a dental adjuvant or a barrier material.

9. A dental care strip and barrier material as claimed in claim 8 that provides a short acting bolus to deliver the barrier material to deep cracks and fissures in the teeth.

10. A dental care strip as claimed in claim 8 in which the barrier material is a calcium salt, cheese or cocoa powder.

11. A dental care strip as claimed in claim 3 which forms a long acting bolus to remove materials in the deep cracks and fissures of the teeth.
